(19) United States
(12) Patent Application Publication
(10) Pub. No.: US 2009/0030481 A1
(43) Pub. Date: Jan. 29, 2009

(54) Implantable Microphone for Treatment of Neurological Disorders

(75) Inventor: Werner Lindenthaler, Oberperfuss (AT)

Correspondence Address: BROMBERG & SUNSTEIN LLP, 125 SUMMER STREET, BOSTON, MA, 02110-1618, US

(73) Assignee: MED-EL Elektromedizinische Geraete GmbH, Innsbruck (AT)

(21) Appl. No.: 12/234,957

(22) Filed: Sep. 22, 2008

Related U.S. Application Data
(63) Continuation-in-part of application No. 11/750,572, filed on May 18, 2007.
(60) Provisional application No. 60/801,350, filed on May 18, 2006.

(51) Int. Cl. A61N 1/36; A61M 37/00

(52) U.S. Cl. 607/48; 604/65

(57) ABSTRACT

A system and method is described for adjunct electrical neuromodulation therapy of neurological disorders. In response to a detected event sensed by an implantable microphone, a targeted component is selectively stimulated by electrical stimulation pulses, or a therapeutically effective amount of a selected drug may be delivered to a targeted physiological function location.

This application is a continuation-in-part of U.S. patent application Ser. No. 11/750,572, filed May 18, 2007, which claimed priority to U.S. Provisional Application 60/801,350, filed May 18, 2006, which are hereby incorporated by reference.

### FIELD OF THE INVENTION

The invention relates to implants for neurological disorders, and specifically to use of an implantable microphone as an afferent part of an implant for treating neurological disorders.

### BACKGROUND ART

The recurrent laryngeal nerve, which innervates the larynx, contains motor fibers that innervate both the abductor/opener and adductor/closer muscles of the vocal folds. Damage to this nerve compromises both of these functions and arrests the vocal fold just lateral to the midline. In unilateral paralysis, the voice is breathy and aspiration can occur because of compromised adduction, but airflow during inspiration is minimally impaired. Adequate ventilation of the lungs is assured because abduction of the opposite fold can still occur with each inspiration. In bilateral paralysis, there is a loss of abductory function in both folds, the voice may be minimally impaired because of fold symmetry and their paramedian position in most of the patients, but airway discomfiture is usually severe. Typically, the patient can tolerate restricted activity or may be relegated to a sedentary lifestyle until treatment is administered. In some situations, however, the condition may be life-threatening.

Clinical management of vocal fold paralysis focuses on the major laryngeal dysfunction associated with each of these two main types. Conventional treatments for unilateral paralysis aim at medialicing the fold to improve voice production. Treatment for bilateral paralysis typically requires a tracheotomy to restore sufficient airflow to the lungs. The tracheotomy is left in place until nerve regeneration and muscle reinnervation has returned. However, in many cases, muscle reinnervation is either incomplete or inappropriate resulting in chronic paralysis. Under such conditions, surgical resection of the vocal fold (i.e., cordotomy) is employed to permanently increase the airway and relieve the patient of his tracheotomy. Although these conventional methods of treatment have been useful, they are less than ideal, since they tend to improve upon one laryngeal function at the expense of another. For example, cordotomy improves ventilation, but compromises voice production and airway protection.

Surgical techniques, such as laser arytenoidectomy and partial cordectomy, can be performed to widen the airway and relieve dyspnea in the case of chronic paralysis. However, these procedures compromise voice and airway protection to restore ventilation through the mouth. They also ignore the long-term effects of ensuing atrophy on vocal fold mass and position. In general, the greater the cartilaginous or membranous resection associated with either technique, the greater the morbidity. A number of modifications of these two strategies have been devised in an attempt to strike a more delicate balance between improved oral ventilation and impaired voice and swallowing. However, a more conservative stance toward resection increases the probability of failed intervention and the necessity for revision surgery. A new, more physiological approach termed laryngeal pacing has been studied in animal models as a means to restore oral ventilation.

Application of FES to paralyzed laryngeal muscles was introduced into human clinical otolaryngology in 1977 by Zealear D L, Dedo H H, Control Of Paralyzed Axial Muscles By Electrical Stimulation, Acta Otolaryngol (Stockholm) 1977, 83:514-27, incorporated herein by reference, which specifically addressed the case of unilateral vocal fold paralysis. Patients normally breathe well, but they cannot approximate both vocal folds. As a result, their voice is weak and breathy, and they tend to aspirate fluids. Zealear and Dedo proposed that a unilaterally paralyzed patient could be reanimated to close appropriately by electrical stimulation triggered by signals relayed from its contralateral partner. As simpler surgical methods were discovered to restore function in unilateral vocal fold paralysis, the development of an implantable neuroprostheses for this condition has not been vigorously pursued.

Mayr, Zrunek, et al., A Laryngeal Pacemaker For Inspiration Controlled Direct Electrical Stimulation Of Denervated Posterior Cricoarytaenoid Muscle In Sheep, Eur. Arch. Otorhinolaryngol, 248(8):445-448, 1991, incorporated herein by reference, described 8 sheep with denervated PCAs which received implants for from 5-18 months, and ruled out reinnervation by control.

Obert et al., Use Of Direct Posterior Cricoarytenoid Stimulation In Laryngeal Paralysis, Arch. Otolaryngol 1984, 110: 88-92, incorporated herein by reference, restored full abduction in bilaterally denervated dogs implanted with single-stranded teflon electrodes, using 20 ms stimulus pulses delivered at 20-40 Hz and 2-3 mA. Their study suggested that stimulus pulses should be synchronized with inspiratory signals in abductor pacing. Bergmann et al., Respiratory Rhythmically Regulated Electrical Stimulation Of Paralyzed Muscles, Laryngoscope, 1984, 94:1376-80, incorporated herein by reference, successfully implanted this idea of respiratory regulation of stimuli, using signals relayed from chest wall expansion. Canine PCA muscles were activated using parameters of 30 Hz, 1 ms, and large amplitudes of up to 50 mA.

Kano and Sasaki, Pacing Parameters of the Canine Posterior Cricoarytenoid Muscle, Ann. Otol. Rhinol. Laryngol., 100:584-588, 1991, incorporated herein by reference, used a pair of coiled electrodes, separated by 2 mm, to stimulate the PCA. They observed promising abductions at 60-90 Hz and 2 ms. Bergmann et al reported 2-3 mm of abduction with stimulation of the PCA using a stimulus delivery system that had been chronically implanted for 11 months.

Otto et al, Coordinated Electrical Pacing Of Vocal Cord Abductors In Recurrent Laryngeal Nerve Paralysis, Otolaryngol. Head Neck Surg., 1985, 93:634-8, incorporated herein by reference, used electromyographic (EMG) signals from the diaphragm to regulate stimuli to denervated canine PCA muscles, and reportedly restored full abduction of the glottis.

Zealear and Herzon, Technical Approach For Reanimation Of The Chronically Denervated Larynx By Means Of Functional Electrical Stimulation, Ann. Otol. Rhinol. Laryngol., 1994 Sep., 103(9):705-12, incorporated herein by reference, first introduced use of tiny coiled electrodes for abductor pacing in a study of inspiratory trigger sources including tracheal elongation, diaphragm EMG signals, phrenic nerve activity, and intrathoracic pressure changes.

Zealear et al, Technical Approach For Reanimation Of The Chronically Denervated Larynx By Means Of Functional Electrical Stimulation, Ann. Otol. Rhinol. Laryngol. 1994, 103: 705-12, incorporated herein by reference, implanted an electrode array 3 months after RLN section, and the paralyzed stump was electro stimulated to rule out reinnervation. The hot spots were located in the middle of the PCA muscle, several millimeters from the median raphe, and covered 30-40% of the muscle surface area.

During chronic pacing, it would be desirable to stimulate above the fusion frequency for the PCA muscle so that a smooth abduction of the vocal cord would be achieved. In each animal, the chronically denervated muscle had a lower fusion frequency than its innervated partner. In a chronic implant, it would be desirable to lower the rate of stimulation under 30 Hz closer to that of the fusion frequency (mean: 21.77 Hz) to conserve charge. FIG. 3 shows views of a clinical patient with laryngeal hemiplegia both at rest and during stimulation with 4.5 mA at 24 Hz. As the pulse duration was increased, the efficiency in activating chronically denervated muscle increased and surpassed that of the innervated muscle at durations greater than 1-2 ms. However above 2 ms, stimulation became less efficient for both muscles because of charge loss through current shunts normally found in tissue. The amount of vocal cord excursion was only 40-70% of that produced with stimulation of the normally innervated muscle, indicative of denervation atrophy and loss of muscle contractility.

Sanders I et al., Arytenoid Motion Evoked By Regional Electrical Stimulation Of The Canine Posterior Cricoarytenoid Muscle, Laryngoscope. 1994 April; 104(4):456-62, incorporated herein by reference, systematically evaluated stimulation delivered to the denervated canine PCA muscles, using single-stranded, stainless steel electrodes 1 cm in length. Measures of abduction were obtained following an overdose of curare designed to mimic vocal fold paralysis via neuromuscular blockade. After RLN section and 2 weeks' time, measures of abduction were repeated in these animals. Results documented 3 mm of vocal cord excursion with 1 ms, 30 Hz, and 1-50 mA.

Sanders I., Electrical Stimulation Of Laryngeal Muscle, Otolaryngol Clin North Am. 1991 October; 24(5): 1253-74, incorporated herein by reference, left 4 dogs undisturbed for 6 months to allow atrophy to occur. After 6 months of atrophy, the responses of the animals had decreased to roughly 60% of initial values. The two dogs that did not undergo stimulation continued to atrophy during the following 4 months to 40% of initial values. The two dogs that underwent electrically induced exercise, however, increased their responses dramatically. Not only had their responses returned to normal, but they were uniformly greater than normal, the average approximately 200% that of their initial denervated state. Gross examination of the excised larynges demonstrated that the stimulated group had maintained muscle bulk while the non-stimulated group was noticeably atrophic. Denervated dog PCA could be stimulated with pulses as short as 2 ms. Any lower, and the needed voltage jumped exponentially. Sanders used similar pulse widths to chronically stimulate denervated muscle for months. This is the minimum and presupposes that the electrode is placed directly adjacent to the muscle.

Zealear DL et al., Reanimation Of The Paralyzed Human Larynx With An Implantable Electrical Stimulation Device, Laryngoscope. 2003 July; 113(7): 1149-56, incorporated herein by reference, reported on four human patients implanted with adapted pain pacemaker systems. In the four patients tested, electromyographic (EMG) motor unit activity was present in the PCA and thyroarytenoid (TA) muscles during voluntary effort. These recordings showed inappropriate firing patterns. For example, inspiratory motor unit activity was recorded from the TA muscle characteristic of a PCA motor unit. In particular, a deep inspiration or sniff increased the rate of firing of individual motor units and enhanced the overall interference response. This inappropriate activity was indicative of synkinetic reinnervation.

In follow-up sessions, the optimum stimulus parameters for vocal fold abduction were studied. A one- to two-second train of one-millisecond pulses delivered at a frequency of 30 to 40 pulses per second (pps) and amplitude of 2 to 7 V effectively produced a dynamic airway. One to two seconds of stimulated abduction allowed sufficient air exchange with each breath. Although a previous study in the canine found 2-millisecond duration as the optimum pulse width for recruiting both reinnervated and non-reinnervated muscle fibers, the maximum pulse width that the stimulator could deliver was 1 millisecond. A frequency of 30 to 40 pps generated a fused, tetanising muscle contraction and a smooth vocal fold abduction with maximum opening. The device was set to deliver an average of 10 stimulus sequences (bursts) every minute to match the patient's respiratory rate at a moderate level of activity. The ideal stimulus amplitude was one that evoked maximum vocal fold opening without inducing discomfort or nociception. At this amplitude, the patient could feel the stimulus, which helped entrain inspiration to the stimulus cycle. Stimulated abduction significantly increased the magnitude of glottal opening in patients 1 to 5 from preoperative levels (P<0.0008). Stimulated glottal opening was large in patients 1, 3, and 4 (3.5-7 mm) and moderate in patient 2 (3 mm). In patient 5, stimulation also produced a large abduction of 4 mm, but the response was delayed in time.

In order to decrease current spread and the high power requirements of FES devices, the placement of electrodes should localize current to the target muscle or nerve (if the muscle is innervated—even if it is synkinetically reinnervated) as much as possible. This may be accomplished by placing the electrodes inside the muscle, or on its surface, a procedure that produces two technical problems: (1) surgical exposure of the muscle causes scarring which eventually decreases muscle mobility; and (2) because electrodes must be close to their target to be efficient, they are exposed to muscle movement. The constant abrasion of the electrode against the muscle breaks the electrode or causes extensive fibrosis in the muscle. This difficulty plagued the early development of the cardiac pacer and persists today in many experiments involving chronic stimulation of denervated muscle, including the denervated PCA. As a result, there has not been a truly successful chronic device for stimulation of denervated muscle.

In 1992 for unilateral vocal cord paralysis, Goldfarb used the electric activity of the healthy side as a trigger for synchronization with breathing and vocalization. See, U.S. Pat. No. 5,111,814. This method is not applicable for the clinically more relevant bilateral paralysis. Lindenthaler described a pacemaker for bilateral vocal cord palsy due to autoparalysis (equivalent to synkinetic Recurrent Laryngeal Nerve (RLN) reinnervation), which is triggered by another muscle or nerve signal that is activated synchronic to breathing, e.g., diaphragm breathing muscles, infrahyoidal muscles of the neck. The pacemaker then stimulates structurally intact but autoparalytic nerve. See, U.S. Pat. No. 7,069,082.

All the sensor signals described have not been successful in animal experiments —especially not in the chronic implanted condition lasting longer than 12 month. This was mainly because of tissue ingrowths and consequently reduction of the signal to noise ratio. So this invention describes an implantable microphone as a sensor for the different phases of respiration. The inspiratory and expiratory airflow has a bottle neck at the vocal folds. A barrier in airflow generates turbulences and they generate a sound—like the pressure on the arteries and the sensed noise of the blood flow for each pulse. Depending on the frequency band of the signal tissue ingrowths is not an important factor for a microphone signal and sound is transmitted trough tissue, so the implanted microphone does not need to be fixed directly in the effected area of the airflow but separated from this region when an appropriate tissue is able to transmit the sound signal.

### SUMMARY OF THE INVENTION

Embodiments of the present invention are directed to using an implantable sensing microphone to generate a sensing input for controlling a physiological function such as vocal fold opening for inspiration, vocal fold closing for speech production, and vocal fold closing for protection against aspiration. The sensing microphone generates an electrical signal that is representative of and responsive to activity at an internal sensing location of a user. For example, for controlling the movement of the vocal folds, the sensing microphone might be positioned to sense activity in the larynx such as near the crycoid or thyroid cartilage, the thorax or the sternum. In specific embodiments, the sensing microphone may monitor pressure and/or distension changes at the internal sensing location, and/or contraction changes of a targeted muscle at the internal sensing location.

A control unit may be coupled to the sensing microphone, and in response to the microphone signal, may generate a stimulation signal such as a sequence of electrical pulses to electrically stimulate a targeted physiological function location. For example, one or more stimulation electrodes may stimulate the vocal fold opening muscle (posterior cricoarytenoid muscle) directly or activate this muscle by stimulating the innervating nerve.

In addition or alternatively, an implantable drug delivery device may be coupled to the sensing microphone, and in response to the microphone signal may deliver a therapeutically effective amount of a selected drug to a targeted physiological function. The sensing microphone generates an electrical signal that is representative of and responsive to activity at an internal sensing location of a user.

For example, the drug delivery device may be a drug delivery pump arrangement and the embodiment may also include a drug delivery catheter for delivering the selected drug to the target physiological function location.

In specific embodiments, the targeted physiological function location includes an afferent function and/or an efferent function. And the embodiment may further be responsive to user control in generating the stimulation signal and/or drug delivery signal. The sensing microphone may specifically monitor pressure changes at the internal sensing location and/or contraction changes of a target at the internal sensing location.

### BRIEF DESCRIPTION OF THE DRAWINGS

FIG. 1 shows one embodiment of the present invention in which a sensing microphone is integrated into the body of an implantable control unit.

FIG. 2 shows another embodiment in which a sensing microphone is physically separate from the implantable control unit.

FIG. 3 shows an example of an embodiment such as the one in FIG. 2 as implanted to monitor and control the functioning of the vocal folds.

FIG. 4 shows an example of measurements in pig experiments with acutely implanted microphones at different locations in the throat.

FIG. 5 shows signals for animal experiments with an accelerometer.

FIG. 6 shows signals for animal experiments with a microphone.

### DETAILED DESCRIPTION OF SPECIFIC EMBODIMENTS

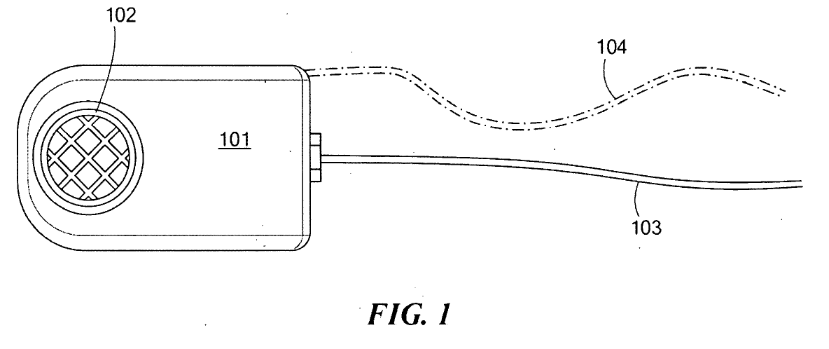

FIG. 1 shows an example of an implantable physiological function control system according to one specific embodiment of the present invention. Implantable control unit 101 contains a built-in sensing microphone 102. The control unit 101 is implanted so that the sensing microphone 102 is able to sense pressure, distension and/or contraction activity at a target internal sensing location such as the wall of the patient's bladder in the lower urinary tract. Another example of the target internal sensing location is along the airway in patients with bilateral vocal cord paralysis for sensing inspiration for vocal fold opening, for vocal fold closing for speech production, and/or for vocal fold closing for protection against aspiration. More specifically, examples of the target internal sensing location include without limitation the synkinetic reinnervated posterior cricoarytenoid muscle such that the stimulation signal applied by the electrode induces muscle contraction, a denervated posterior cricoarytenoid muscle such that the stimulation signal applied by the electrode induces muscle contraction, and/or a vocal fold closing muscle such that the stimulation signal applied by the electrode induces muscle contraction. Other physiological functions that may usefully be monitored and/or controlled include without limitation overactive bladder, urinary urge incontinence, urinary urge-frequency incontinence, urinary urge retention, micturition, fecal incontinence, defecation, peristalsis, pelvic pain, prostatitis, prostatalgia and prostatodynia, erection, and ejaculation.

The microphone senses such activity and generates a representative electrical signal for the control unit 101. Rather than a microphone as such, some embodiments may use other similar types of sensor such as, without limitation, a piezoelectric pressure sensor or other pressure sensor. In response, the control unit may generate a stimulation signal for a stimulation electrode such as in an electrode array 103 to electrically stimulate a targeted location such as the patient's urethra or the patient's vocal cords. For example, the stimulation signal may be a sequence of electrical pulses for the electrodes to stimulate the target location. In addition, or alternatively, the control unit 101 may use an implanted drug delivery catheter 104 supplied by an implanted drug delivery pump to deliver a therapeutically effective amount of a selected drug to a target location such as the patient's airway or urethra. In specific embodiments, the operation of the control unit 101 may be responsive to volitional control of the patient.

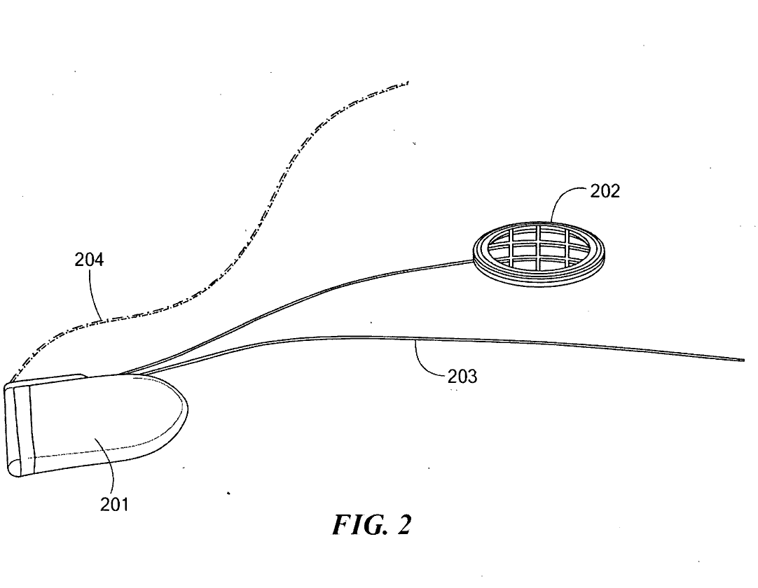

FIG. 2 shows an alternative embodiment in which an implantable sensing microphone 202 is physically separate from the control unit 202. This allows for sensing microphone 202 to placed at an sensing location which is optimal for detecting the target activity, while the control unit 201 can be implanted at a different location which may be more convenient in terms of its bulk and positioning, and may for example, allow for more convenient post-implantation servicing of the control unit 201 without disturbing the sensing microphone 202 and its location.

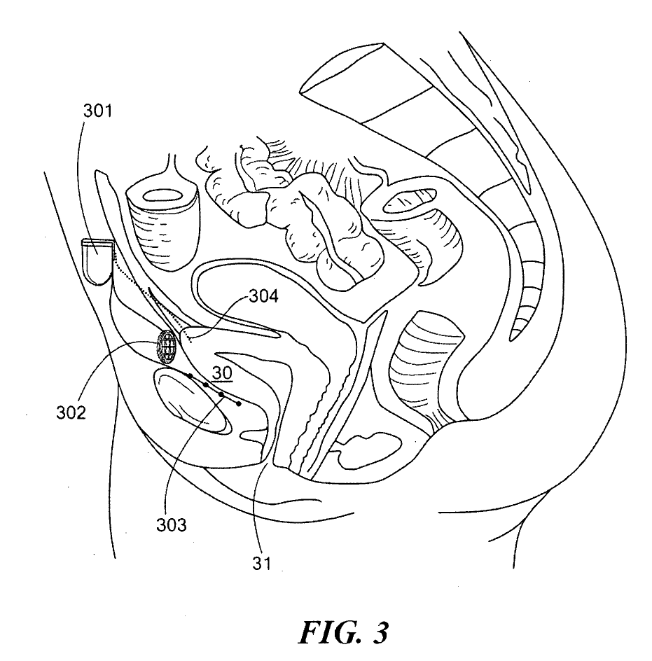

FIG. 3 shows an example of an implanted system such as the one shown in FIG. 2 for monitoring and controlling the patient's bladder 30 and urethra 31. In FIG. 3, an implantable sensing microphone 302 is located near the wall of the patient's bladder 30 and senses activity therein. A representative electrical signal from the sensing microphone 302 is coupled to a control unit 301 which acts responsively. For example, the control unit 301 may generate a stimulation signal for one or more stimulation electrodes such as in an implanted electrode array 303 to stimulate the bladder 30 and/or the urethra 31. In addition or alternatively, the control unit 301 may cause implanted drug delivery catheter 304 to deliver a therapeutically effective amount of one or more selected drugs to a target location such as the interior volume of the bladder 30.

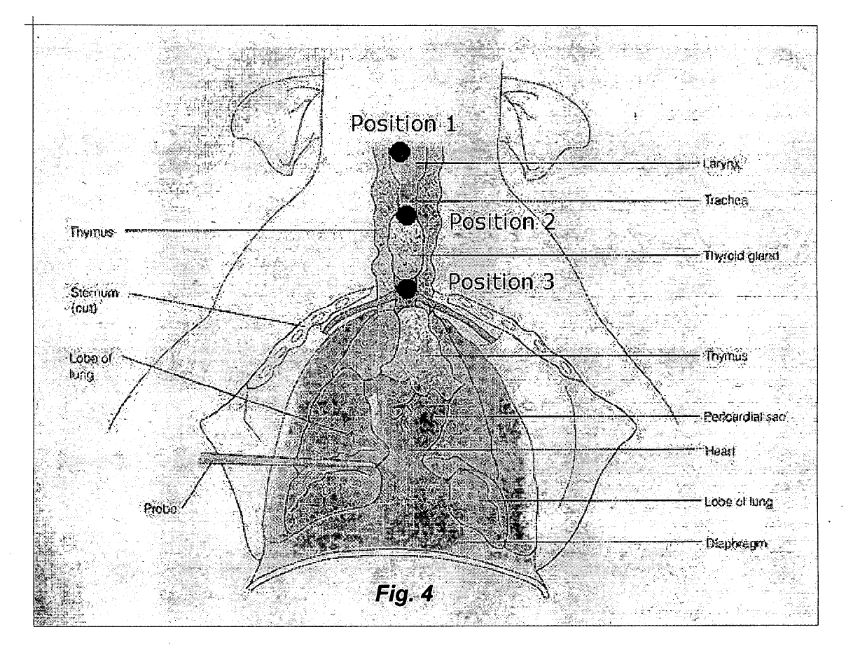

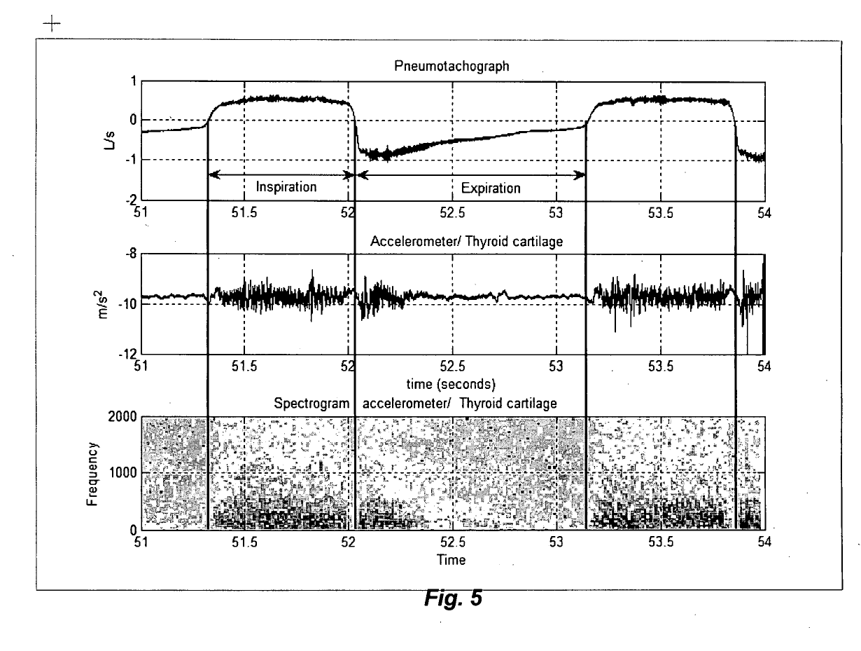

FIG. 4 shows an example of measurements in pig experiments with acutely implanted microphones at different locations in the throat, to monitor, for example, the larynx, trachea, and thyroid gland. In this case, a MEMS accelerometer was used which contained a seismic mass connected with a spring to the housing. The seismic mass and the spring may be created in an etching process out of mono-crystalline silicon structure. An applied acceleration causes a deflection of the seismic mass which is measured with a capacitive method with a sensing element having two parallel plate capacitors acting in a differential mode. The signal of the detection circuit can be internally amplified and low-pass filtered with external capacitors. A pneumotachograph signal is shown in the upper tracing of FIG. 5 for an accelerometer placed on the thyroid cartilage to measure the flow rate of the inhaled and exhaled air. Flow rates above zero occur during inhalation while signal below zero designate expiration. The second and third tracings from the top of FIG. 5 show the signal of the accelerometer in the time and frequency domain.

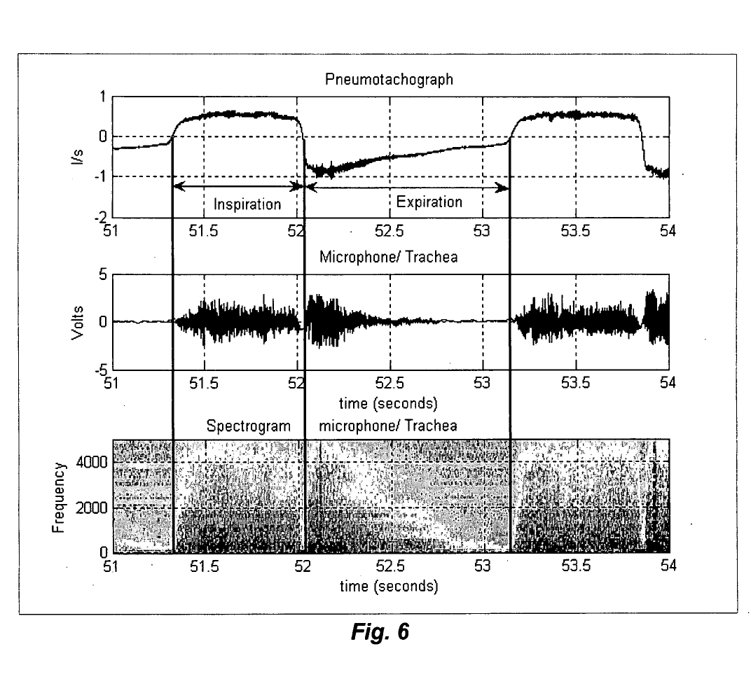

In other animal experiments, highly sensitive electret microphones were used to detect the breathing signals. An electret microphone is a kind of condenser microphone with a permanent charged material between the plates of the condenser and the microphone signals were amplified and band-pass filtered for data acquisition. In FIG. 6, the upper tracings show the signal of the pneumotachograph which measured the flow rate of the inhaled and exhaled air. Flow rates above zero occur during inhalation while signal below zero designate expiration. The second and third tracings from the top of FIG. 6 show the signal of the microphone in the time and frequency domain. For the measurements, the microphones were placed in different the region of the larynx—here the measurements for placing the microphone on the trachea are shown. In the frequency domain, the signals of the microphone and accelerometer show a broad band noise with a rapid decrease between 800 Hz and 1500 Hz during the whole inspiration phase. During expiration, over time an amplitude decrease at higher frequencies can be recognized. Breathing pauses between the two respiration phases can be seen as short time intervals without signals above 100 Hz.

In specific embodiments, the targeted physiological function location may be an afferent function such as a nerve sensing location, and/or an efferent function such as a motor nerve location. Specific embodiments may seek to exploit spinal inhibitory systems that can interrupt a detrusor contraction by electrically stimulating afferent anorectal branches of the pelvic nerve, afferent sensory fibers in the pudendal nerve, and/or muscle afferents from the limbs.

Although various exemplary embodiments of the invention have been disclosed, it should be apparent to those skilled in the art that various changes and modifications can be made which will achieve some of the advantages of the invention without departing from the true scope of the invention.

What is claimed is:

1. A system for controlling a physiological function comprising:
an implantable sensing microphone for generating a microphone signal representative of activity at an internal sensing location of a user; and
a control unit coupled to the sensing microphone and responsive to the microphone signal for generating a stimulation signal to electrically stimulate a targeted physiological function location.

2. A system according to claim 1, wherein the stimulation signal includes a sequence of electrical pulses.

3. A system according to claim 1, further comprising:
at least one implantable stimulation electrode for applying the stimulation signal to the targeted physiological function location.

4. A system according to claim 3, wherein the targeted physiological function location includes a synkinetic reinnervated posterior cricoarytenoid muscle such that the stimulation signal applied by the electrode induces muscle contraction.

5. A system according to claim 3, wherein the targeted physiological function location includes a denervated posterior cricoarytenoid muscle such that the stimulation signal applied by the electrode induces muscle contraction.

6. A system according to claim 3, wherein the targeted physiological function location includes a vocal fold closing muscle such that the stimulation signal applied by the electrode induces muscle contraction.

7. A system according to claim 1, wherein the control unit is further responsive to user control in generating the stimulation signal.

8. A system according to claim 1, wherein the physiological function includes at least one of vocal fold opening for inspiration, vocal fold closing for speech production, and vocal fold closing for protection against aspiration.

9. A system according to claim 1, wherein the internal sensing location is along the airway of the user.

10. A system according to claim 1, wherein the sensing microphone monitors pressure changes at the internal sensing location.

11. A system according to claim 1, wherein the sensing microphone monitors contraction changes of a target at the internal sensing location.

12. A system according to claim 1, wherein the sensing microphone monitors distension changes of a target at the internal sensing location.

13. A system for controlling a physiological function comprising:
an implantable sensing microphone for generating an electrical signal representative of activity at an internal sensing location of a user; and
an implantable drug delivery device coupled to the microphone and responsive to the microphone signal for delivering a therapeutically effective amount of a selected drug to a targeted physiological function location.

14. A system according to claim 13, further comprising:
a drug delivery catheter for delivering the selected drug to the targeted physiological function location.

15. A system according to claim 13, wherein the drug delivery device is an implanted drug delivery pump.

16. A system for monitoring a physiological function comprising:
an implantable sensing microphone for generating an electrical signal representative of activity at an internal sensing location of a user; and
a control unit coupled to the microphone.

17. A system according to claim 16, wherein the internal sensing location is at a position along the airway of the user.

18. A system according to claim 16, wherein the sensing microphone monitors pressure changes at the internal sensing location.

19. A system according to claim 16, wherein the sensing microphone monitors contraction changes of a target at the internal sensing location.

20. A system according to claim 16, wherein the sensing microphone monitors distension changes of a target at the internal sensing location.

21. A system according to claim 16, wherein the control unit is further responsive to user control.

22. A system according to claim 16, wherein the activity includes at least one of vocal fold opening for inspiration, vocal fold closing for speech production, and vocal fold closing for protection against aspiration.
